(19) United States
(12) Patent Application Publication
(10) Pub. No.: US 2014/0243779 A1
(43) Pub. Date: Aug. 28, 2014

(54) Modular Body-Adhering Absorbent Article

(71) Applicant: KIMBERLY-CLARK WORLDWIDE, INC., Neenah, WI (US)

(72) Inventor: Hwa Young Kim, Greenville, WI (US)

(73) Assignee: Kimberly-Clark Worldwide, Inc, Neenah, WI (US)

(21) Appl. No.: 13/779,890

(22) Filed: Feb. 28, 2013

(51) Int. Cl. A61F 13/82

(52) U.S. Cl. CPC A61F 13/82
USPC 604/401

(57) ABSTRACT

An absorbent article system includes a support member having anterior and posterior ends, a first anchor connection zone adjacent one of the anterior and posterior ends, and an absorbent article component connection zone between the anterior and posterior ends. The system also includes a first anchor configured to be connectable to the first anchor connection zone, wherein one of the first anchor and the first anchor connection zone includes an anchor attachment mechanism, wherein the first anchor has a body-facing side and an attachment side, the body-facing side including a body adhesive, and a second anchor connection zone adjacent the other of the anterior and posterior ends, and a second anchor configured to be connectable to the second anchor connection zone, wherein the second anchor has a body-facing side and an attachment side, the body-facing side including a body adhesive.

### BACKGROUND

Disposable absorbent articles for the absorption and containment of urine, menses and other body exudates are generally known in the art. Generally, these articles are referred to as absorbent personal care articles and have taken various forms including, diapers for infants and children, training pants for children, sanitary napkins, pantiliners, incontinence pads, incontinence garments and the like, for teenagers and adults. Of these absorbent personal care articles, sanitary napkins, incontinence pads and pantiliners are articles that are not garment-like in form, meaning that they do not rely on the body structure of the user to stay in place during use. For example, diapers use the hips and waist of the user to hold the diaper in place during use. As a result, sanitary napkins, pantiliners and incontinence pads are less noticeable and more discrete than diapers and incontinence garments.

Instead, sanitary napkins, pantiliners, and incontinence pads use an undergarment attaching device to hold the article in place during use. Conventional means of holding these absorbent personal care articles in place include, for example, garment attachment adhesives, which are generally pressure sensitive adhesives; attachment panels, which are sometimes referred to as wings or flaps, extending from the longitudinal side edges of the absorbent article that wrap around the undergarment of the user; or a body adhesive, which secures the absorbent personal care article directly to the body of a user. In addition, combinations of the attachment devices have also been used.

In selecting an attachment device, several considerations must be considered and balanced. The attachment device must hold the absorbent personal care article in place, providing adequate protection, the attachment device must be comfortable, and the attachment device must be convenient and easy to use. Currently, the most common attachment device is a garment adhesive. While absorbent personal care articles with the garment adhesive attachment device have performed well, remaining in place and providing the user with ease of placement and removal, these absorbent personal care articles have suffered from certain drawbacks. For example, the inner crotch surface of the undergarment to which the absorbent personal care articles are adhered is constantly being distorted, twisted and stretched due to the movements of the wearer. As a result, frequently the garment adhesive detaches with the undesirable result of the sanitary napkin, pantiliner or incontinence pad moving out of position. In an extreme case, detachment of the adhesive can also result in the adhesive folding over on itself and then becoming unavailable for reattachment to the undergarment of the user.

In the case of sanitary napkins, some sanitary napkins have been provided with attachment panels that, in use, are generally folded around the crotch portion of the undergarment and affixed to the outer crotch portion. Although such attachment panels have been partially successful in protecting certain regions of the wearer's undergarment, such sanitary napkins are still subject to the forces that cause the sanitary napkin to be distorted, twisted or stretched.

There is a need in the art for an absorbent personal care article, in the form of a sanitary napkin, incontinence pad or pantiliner, that can be effectively held into place against a user's body, providing an effective means to hold the absorbent personal care article in place, thereby providing adequate protection to the user, providing comfort to the user and providing an attachment device that is convenient and easy to use.

### SUMMARY

It has now been discovered that a modular absorbent article system can be provided with a body-attachable article without body adhesive on the absorbent. In one aspect, the present disclosure describes an absorbent article system including a support member having anterior and posterior ends, a first anchor connection zone adjacent one of the anterior and posterior ends, and an absorbent article component connection zone between the anterior and posterior ends. The system also includes a first anchor configured to be connectable to the first anchor connection zone, wherein one of the first anchor and the first anchor connection zone includes an anchor attachment mechanism, wherein the first anchor has a body-facing side and an attachment side, the body-facing side including a body adhesive.

In another aspect of the present disclosure, an absorbent article system includes a support member having anterior and posterior ends, a first anchor connection zone adjacent one of the anterior and posterior ends, a second anchor connection zone adjacent the other of the anterior and posterior ends, and an absorbent article component connection zone between the anterior and posterior ends; and s first anchor configured to be connectable to the first anchor connection zone, wherein one of the first anchor and the first anchor connection zone includes an anchor attachment mechanism, wherein the first anchor has a body-facing side and an attachment side, the body-facing side including a body adhesive. The system also includes a second anchor configured to be connectable to the second anchor connection zone, wherein one of the second anchor and the second anchor connection zone includes an anchor attachment mechanism, wherein the second anchor has a body-facing side and an attachment side, the body-facing side including a body adhesive, wherein one of the first and second anchors is configured to be connected to the support member by a wearer; and an absorbent article component, wherein one of the absorbent article component and the absorbent article component connection zone includes a pad attachment mechanism, and wherein one of the absorbent article component and the absorbent article component connection zone is configured to allow the absorbent article component to be connected to the support member by a wearer.

In still another aspect of the present disclosure, a system for converting a garment-attached absorbent article to a body-attached absorbent article includes a support member having anterior and posterior ends, a first anchor connection zone adjacent one of the anterior and posterior ends, a second anchor connection zone adjacent the other of the anterior and posterior ends, and an absorbent article component connection zone between the anterior and posterior ends, the absorbent article component connection zone configured to allow the garment-attached absorbent article to be connected to the support member by a wearer. The system also includes a first anchor configured to be connectable to the first anchor connection zone, wherein one of the first anchor and the first anchor connection zone includes an anchor attachment mechanism, wherein the first anchor has a body-facing side and an attachment side, the body-facing side including a body adhesive.

### DESCRIPTION OF THE DRAWINGS

The foregoing and other features and aspects of the present disclosure and the manner of attaining them will become more apparent, and the disclosure itself will be better understood by reference to the following description, appended claims and accompanying drawings, where:

FIG. 1 is a perspective view of a body-facing side of a body-adhering absorbent article according to one aspect of the present disclosure.

Repeat use of reference characters in the present specification and drawings is intended to represent the same or analogous features or elements of the present disclosure. The drawing is representational and is not necessarily drawn to scale. Certain proportions thereof might be exaggerated, while others might be minimized.

### DETAILED DESCRIPTION

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

“Absorbent structure” refers to the central fluid handling portion of an absorbent article and can include but is not limited to one or more of the following components: cellulose fluff, superabsorbent material, coform, absorbent sponge material, surge material, or wicking material but does not include a topsheet or backsheet.

“Absorbent component” can include but is not limited to one or more of the following components: an absorbent structure, as defined above, a fluid pervious topsheet, a fluid impervious backsheet, or attachment adhesive.

As used herein, the terms “attach” and “affix” and their derivatives refer to the joining, adhering, connecting, bonding, sewing together, or the like, of two elements. Two elements will be considered to be attached or affixed together when they are integral with one another or attached directly to one another or indirectly to one another, such as when each is directly attached to intermediate elements. “Attach” and its derivatives include permanent, releasable, or a refastenable attachment. In addition, the attachment can be completed either during the manufacturing process or by the end wearer.

As used herein, the term “body-facing” means that surface or side of the article that is intended to be disposed toward or placed adjacent to the body of the wearer during ordinary use. The term “garment-facing” means that surface or side that is on the opposite side of the article from the body-facing surface or side. The garment-facing surface is an outward surface of the article and is intended to be disposed to face away from the wearer's body during ordinary use. The garment-facing surface is generally arranged to face toward or placed adjacent to the wearer's undergarments or clothing when the article is worn.

As used herein, the terms “connected” and “coupled” are intended to mean directly connected and indirectly connected. By directly connected or coupled, it is intended that the connected elements are in contact with one another or affixed to one another. By indirectly connected, it is intended that one or more intervening or intermediate elements are between the two elements that are secured or “connected” together. The intervening elements can be affixed.

As used herein, the term “standard pad” refers to any pad-like feminine absorbent article readily available in a retail setting and intended to be connected to a wearer's garments or undergarments. A “standard pad attachment mechanism” is the means by which a standard pad can be connected to a wearer's undergarments or other structure. Typical standard pad attachment mechanisms include adhesive and mechanical fasteners.

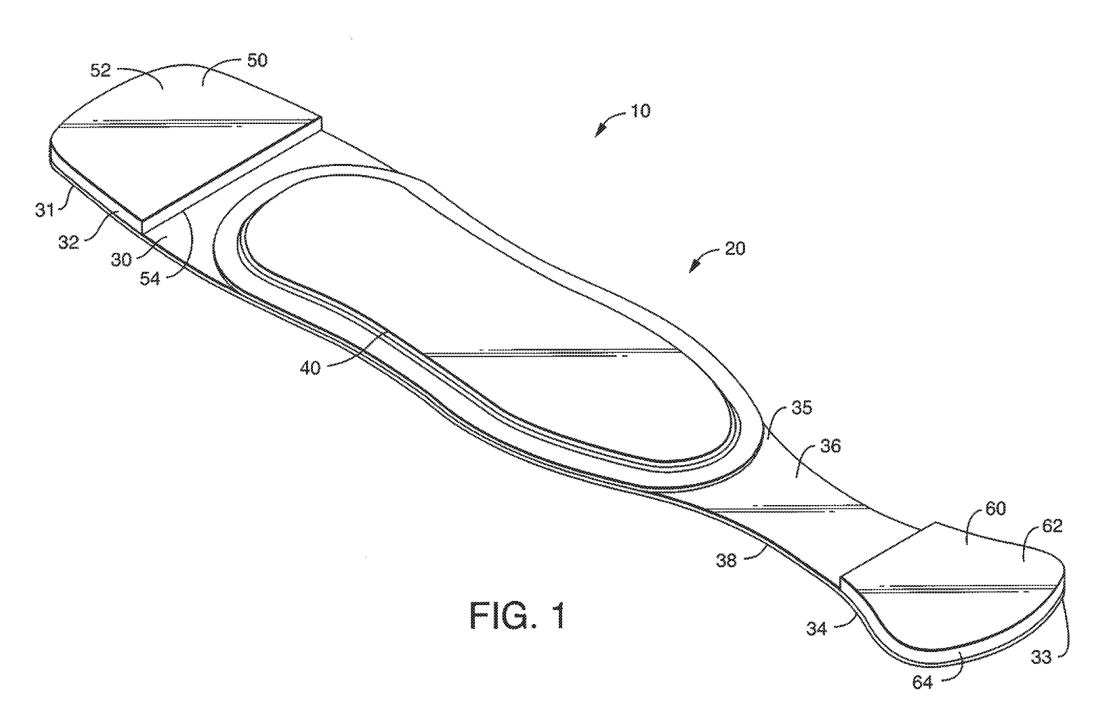

Generally, the present disclosure relates to a modular absorbent article system 10, specifically a modular body-adhering absorbent article 20. Various arrangements and components of the body-adhering absorbent article 20 are described. Now with reference to FIG. 1, one aspect of a body-adhering absorbent article 20 is illustrated. Body-adhering absorbent article 20 includes a support member 30, an absorbent article component 40, a first anchor 50, and a second anchor 60. The body-adhering absorbent article 20 also includes mechanisms by which the various components can be connected.

The absorbent article system 10 of the present disclosure includes a support member 30 having an anterior end 32 and an opposed posterior end 34. The support member 30 also has a body-facing side 36 and a garment-facing side 38. The support member 30 includes a first anchor connection zone 31 adjacent to one of the anterior and posterior ends 32, 34, and a second anchor connection zone 33 adjacent the other of the anterior and posterior ends 32, 34. In the aspect illustrated in FIG. 1, the first anchor connection zone 31 is disposed adjacent the anterior end 32, and the second anchor connection zone 33 is disposed adjacent the posterior end 34.

The support member 30 is preferably a flexible material that can be formed into a continuous web. Suitable support member materials include nonwovens and papers as well as combinations of the same with cotton fabrics, viscose fabrics, cotton-viscose blended fabrics, synthetic fiber wovens, and synthetic fiber nonwovens. Suitable as synthetic fibers are, in particular, polyolefins, polyamides and polyurethane. Particularly well-suited support member materials include nonwoven fabrics such as air laid webs of natural or synthetic fibers or blends thereof. The layer thickness of the carrier is preferably 0.1 to 10 mm, preferably 0.5 to 5 mm. The support member 30 can be absorbent or non-absorbent. The support member 30 can be reusable, durable, or disposable, and can be elastomeric. The support member 30 can also be configured such that it is customizable in width, length, and/or shape through the use of perforations, printed cut lines, or by any other suitable method.

The absorbent article system 10 of the present disclosure also includes a first anchor 50 having a body-facing side 52 and a garment-facing side 54. The first anchor 50 is configured to be connectable to the first anchor connection zone 31 through the use of an anchor attachment mechanism. The anchor attachment mechanism can be disposed on the first anchor connection zone 31, or on the garment-facing side 54 of the first anchor 50, or both. The first anchor 50, when connected to the support member 30, provides a body-facing side 52 in an orientation conducive to connection to the wearer's body. This connection can be enhanced through the use of a body adhesive on the body-facing side 52 such that the first anchor 50 is connected to the body of the wearer and to the first anchor connection zone 31 of the support member 30.

The absorbent article system 10 of the present disclosure can also include a second anchor 60 having a body-facing side 62 and a garment-facing side 64. The second anchor 60 is configured to be connectable to the second anchor connection zone 33 through the use of an anchor attachment mechanism. The anchor attachment mechanism can be disposed on the second anchor connection zone 33, or on the garment-facing side 64 of the second anchor 60, or both. The second anchor 60, when connected to the support member 30, provides a body-facing side 62 in an orientation conducive to connection to the wearer's body. This connection can be enhanced through the use of a body adhesive on the body-facing side 62 such that the second anchor 60 is connected to the body of the wearer and to the second anchor connection zone 33 of the support member 30.

The first and second anchors 50, 60 can be of any suitable shape, design, or material. The first and second anchors 50, 60 can also be identical, interchangeable, or both. The first and second anchors 50, 60 can be absorbent or non-absorbent. The first and second anchors 50, 60 can be reusable, durable, or disposable, and can be elastomeric. The first and second anchors 50, 60 can also be configured such that they are customizable in width, length, and/or shape through the use of perforations, printed cut lines, or by any other suitable method.

The anchor attachment mechanism or mechanisms employed in the system 10 can be permanently fastenable, refastenable, or provide for the first and second anchors 50, 60 to be affixed to the support member 30. The first and/or second anchors 50, 60 can be connected to the support member 30 by a manufacturer or by the wearer. The first and second anchors 50, 60 can be configured such that the connection to the support member 30 can be made at any desired point within the respective anchor connection zones 31, 33 to accommodate different wearer physiologies, garments, and comfort levels, among other factors influencing how a wearer prefers the system 10 to be arranged.

The anchor attachment mechanism can include an adhesive, a mechanical fastener, any other suitable attachment mechanism, or any combination of these. The attachment can be in a permanent manner, meaning that the first and second anchors 50, 60 are generally intended not to be removable by the wearer of the body-adhering absorbent article 20. Alternatively, the first and second anchors 50, 60 can be constructed to be removable by the wearer, meaning that the first and second anchors 50, 60 can be removed and replaced with other first and second anchors 50, 60 by the wearer of the body-adhering absorbent article 20, or be replaced with nothing at all. In some aspects, when the first and second anchors 50, 60 are attached to the support member 30 in a permanent manner, meaning that the first and second anchors 50, 60 are not intended to be removed by the wearer, various bonding means can be used, such as a construction adhesive for example. Examples of useable construction adhesives include any adhesive that will effectively hold the first and second anchors 50, 60 in place, so as not to be separated from the support member 30. Commercially available construction adhesives usable in the present disclosure include, for example, REXTAC brand adhesives available from Huntsman Polymers, Houston, Tex. Other means can be used to hold the first and second anchors 50, 60 to the support member 30 including suitable bonding techniques, including, but not limited to, adhesive bonds, cohesive bonds, thermal bonds, ultrasonic bonds, embossing, crimping, entangling, fusing, hook and loop, or the like, and combinations thereof.

Where the first and second anchors 50, 60 are preferably removably attached, the first and second anchors 50, 60 are held in place on the support member 30 by a means that will allow the wearer to remove the first and second anchors 50, 60. One such means of holding the first and second anchors 50, 60 is by using a pressure sensitive adhesive. Suitable pressure sensitive adhesives include, but are not limited to, any commercially available pressure sensitive adhesive. Examples of suitable pressure sensitive adhesives usable to removably hold the first and second anchors 50, 60 in place on the support member 30 include pressure sensitive adhesives available from National Starch, Bridgewater, N.J.

In one aspect of the present application, the first anchor 50 is affixed to the support member 30 by a manufacturer, and the second anchor 60 is connected to the support member 30 by a wearer. In another aspect, both the first and second anchors 50, 60 are provided to the wearer to be placed by the wearer where desired. In yet another aspect, the manufacturer connects the first and second anchors 50, 60 to the support member 30 with the instructions to the wearer that the first and/or second anchors 50, 60 can be disconnected and then re-connected by the wearer as desired.

In another aspect of the present disclosure, one or both of the first and second anchors 50, 60 can be eliminated by applying a body adhesive directly to part or all of the body-facing side 36 of the support member 30.

The support member 30 also includes an absorbent article component connection zone 35 disposed on the body-facing side 36 of the support member 30 between the anterior and posterior ends 32, 34. In various aspects of the present disclosure, the first and second anchor connection zones 31, 33 and the absorbent article component connection zone 35 can be of any size or position, and can overlap one or more of the other zones.

The system 10 includes an absorbent article component 40 configured to be connected to the support member 30 at the absorbent article component connection zone 35. One or both of the absorbent article component 40 and the absorbent article component connection zone 35 includes a pad attachment mechanism to accommodate the connection of the absorbent article component 40 to the absorbent article component connection zone 35.

The pad attachment mechanism or mechanisms employed in the system 10 can be permanently fastenable, refastenable, or provide for the absorbent article component 40 to be affixed to the support member 30. The absorbent article component 40 can be connected to the support member 30 by a manufacturer or by the wearer. The absorbent article component 40 can be configured such that the connection to the support member 30 can be made at any desired point within the respective absorbent article component connection zone 35 to accommodate different wearer physiologies, garments, and comfort levels, among other factors influencing how a wearer prefers the system 10 to be arranged.

In one aspect of the present application, the absorbent article component 40 is affixed to the support member 30 by a manufacturer. In another aspect, the absorbent article component 40 is provided to the wearer to be placed by the wearer where desired. In yet another aspect, the manufacturer connects the absorbent article component 40 to the support member 30 with the instructions to the wearer that the absorbent article component 40 can be disconnected and then re-connected by the wearer as desired. In still another aspect, the system 10 can be configured such that the absorbent article component connection zone 35 can accommodate a standard absorbent pad provided by the wearer, including accommodating a standard pad attachment mechanism.

The attachment can be in a permanent manner, meaning that the absorbent article component 40 is generally intended not to be removable by the wearer of the body-adhering absorbent article 20. Alternatively, the absorbent article component 40 can be constructed to be removable by the wearer, meaning that the absorbent article component 40 can be removed and replaced with another absorbent article component 40 by the wearer of the absorbent article 20, or be replaced with nothing at all. In some aspects, when the absorbent article component 40 is attached to the support member 30 in a permanent manner, meaning that the absorbent article component 40 is not intended to be removed by the wearer, various bonding means can be used, such as a construction adhesive for example. Examples of useable construction adhesives include any adhesive that will effectively hold the absorbent article component 40 in place, so as not to be separated from the support member 30. Commercially available construction adhesives usable in the present disclosure include, for example, REXTAC brand adhesives available from Huntsman Polymers, Houston, Tex. Other means can be used to hold the absorbent article component 40 to the support member 30 including suitable bonding techniques, including, but not limited to, adhesive bonds, cohesive bonds, thermal bonds, ultrasonic bonds, embossing, crimping, entangling, fusing, hook and loop, or the like, and combinations thereof.

Where the absorbent article component 40 is preferably removably attached, the absorbent article component 40 is held in place on the support member 30 by a means that will allow the wearer to remove the absorbent article component 40. One such means of holding the absorbent article component 40 is by using a pressure sensitive adhesive. Suitable pressure sensitive adhesives include, but are not limited to, any commercially available pressure sensitive adhesive. Examples of suitable pressure sensitive adhesives usable to removably hold the absorbent article component 40 in place on the support member 30 include pressure sensitive adhesives available from National Starch, Bridgewater, N.J.

The absorbent article component 40 can also be configured such that it is customizable in width, length, and/or shape through the use of perforations, printed cut lines, or by any other suitable method. The absorbent article component 40 can also include wings such as those provided in some standard pads to assist with or provide the connection means between the absorbent article component 40 and the support member 30.

In certain aspects it can be advantageous for the absorbent article component 40 to have a backsheet and more preferably a fluid impervious backsheet. The backsheet can serve to provide liquid impermeability for the absorbent article component 40, such that any fluids entering the absorbent structure will not flow through the structure to the clothing of a wearer. One example of a commercially available fluid impervious backsheet is the XP-3473a baffle available from Huntsman Packaging Corporation, Houston, Tex.

The absorbent article component 40 can further include an absorbent structure and a fluid pervious topsheet. The absorbent structure can include wicking layers, which can be formed from meltblown microfiber such as the 50 gsm meltblown fibers commercially available from Yuhan-Kimberly Ltd., Korea. The absorbent structure can further include an absorbent layer and an intake layer. The absorbent layer can include one or more layers of absorbent materials, such as fibrous materials and/or superabsorbent materials for example. Each of the layers can include similar materials or different materials. Materials that can be used to form the absorbent layer include those materials conventionally used in absorbent articles and includes materials, such as, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A desired material is wood pulp fluff, for it is low in cost, relatively easy to form, and has good absorbency.

The fluid pervious topsheet should be able to manage different body excretions depending on the type of product. In feminine care products, often the body-side liner or topsheet must be able to handle menses and urine. In certain aspects the topsheet can include a layer constructed of any operative material, and can be a composite material. For example, the body-side liner or body-contacting layer can include a woven fabric, a nonwoven fabric, a polymer film, a film-nonwoven fabric laminate or the like, as well as combinations thereof.

The system 10 can include an additional second support member (not shown) configured to be connectable to the first and/or second anchors 50, 60 and to an absorbent article component 40. The second support member can be identical to or different from the support member 30, and is disposed generally parallel to the support member 30.

In addition, the body-adhering absorbent article 20 can include one or more peel strips (not shown) or other mechanisms configured to shield the body adhesive on the body-facing sides 52, 62 of the first and second anchors 50, 60 from prematurely adhering to anything. In use the wearer removes the peel strip to allow the exposed body adhesive to adhere the body-adhering absorbent article 20 to the wearer's body. Each of the first and second anchors 50, 60 can be provided with its own peel strip.

Construction adhesives used in the manufacture of the body-adhering absorbent article 20 can be any adhesive suitable for adhering two non-woven materials or an elastic material to a non-woven material. Particularly preferred construction adhesives include hot melt adhesives. Preferably the hot melt adhesives are soft and suitable for elastic attachment of elastic material to disposable substrates such as non-woven materials and polyethylene. Examples of appropriate hot melt adhesives are those commonly used in the industry for adhering an elastic material to a non-woven material. Particularly preferred examples of suitable adhesives include, EASYMELT™ (National Starch Chemical Co., USA), HL-8130-E 2P (H.B Fuller Company, USA) and Sanicare SH4-187 HV™ (Henkel, USA). Other commercially available construction adhesives usable in the present disclosure include, for example REXTAC brand adhesives available from Huntsman Polymers of Houston, Tex., as well as adhesives available from Bostik Findley, Inc., of Wauwatosa, Wis. A particularly-preferred construction adhesive is EASYMELT 34-5610 brand adhesive.

As stated above, the body adhesive enables the connection of the body-adhering absorbent article 20 to a wearer. Stated another way, the first and second anchors 50, 60 are the portions of the body-adhering absorbent article 20 that are connected to the body of the wearer. Preferably the body adhesive contacts skin and hair, if present. Preferably the first anchor 50 of the body-adhering absorbent article 20 contacts the region just above the wearer's pubic bone, and the second anchor 60 of the body-adhering absorbent article 20 contacts the region just above the wearer's coccyx, thereby supporting and holding the body-adhering absorbent article 20 and therefore the absorbent article component 40 against the body of the wearer during use. The body adhesive can overlie a portion of the body-facing sides 52, 62 of the first and second anchors 50, 60 or it can overlie the entirety of the body-facing sides 52, 62 of the first and second anchors 50, 60. Generally, the body adhesive will be present as a continuous coating on the body-facing sides 52, 62 of the first and second anchors 50, 60. The body adhesive can also be placed in a pattern. The body adhesive can be applied using any suitable process including inkjet printing, screen printing, extruding the body adhesive from one or more nozzles, slot coating, and the like.

Generally, any suitable pressure sensitive adhesive can be used for the body adhesive provided that the pressure sensitive adhesive is not a known irritant to human skin or that the adhesive is so aggressive that it causes pain to the wearer when the body-adhering absorbent article 20 is removed from the skin. It is also desirable that the adhesive is selected such that the adhesive does not leave a substantial amount of residue on the skin of the wearer when the body-adhering absorbent article 20 is removed. Particularly suitable pressure sensitive adhesive materials are disclosed in U.S. Pat. Nos. 6,213,993 and 6,620,143, both of which are incorporated herein in a manner consistent with the present disclosure. Other suitable adhesives are disclosed in U.S. Pat. Nos. 5,618,281 and 6,316,524, both of which are incorporated herein in a manner consistent with the present disclosure. Additional examples of pressure sensitive adhesives include hydrogels, hydrocolloids, acrylics-based adhesives, and rubber-based adhesives such as Kraton-based adhesives.

In certain aspects the body adhesive preferably includes a UV-curable polymer. As used herein, the term “UV-curable polymer” generally refers to one or more polymers including a photoinitiator that is covalently bound to the polymer. Preferred are polymers including an acrylic polymer backbone molecule that is modified with polymerized photoreactive groups, e.g., a modified benzophenone group that is chemically bonded to the acrylic polymer chain. The polymer is crosslinked by chemical grafting caused by the excitation of the photoinitiator by UV irradiation.

UV curable polymers are commercially available from a number of suppliers and are typically a solvent- and water-free acrylic raw material that can be used for the production of pressure sensitive tapes and labels. These polymers are nearly solid material at room temperature and have to be heated to a temperature of about 120-130° C. to become fluid enough for the coating process. At this temperature, they can be applied with conventional hot melt coating systems. Thus they are processed as hot melts. After being coated on the carrier, the polymer film is crosslinked by UV-irradiation to produce the adhesive properties required.

The body adhesive can be disposed on the body-facing sides 52, 62 of the first and second anchors 50, 60 in an open pattern or a closed pattern. “Open pattern” means that the adhesive can have an intermittent or discontinuous pattern that does not substantially encircle or enclose portions of the body-facing sides 52, 62. An example of an “open” pattern of the adhesive is individual beads of adhesive applied in a discontinuous fashion. “Closed pattern” means the adhesive creates enclosed regions surrounded by adhesive. An example of a closed adhesive pattern is an adhesive overlapping swirl pattern applied in a continuous pattern. The weights of adhesives are limited to less than about 800 grams per square meter, and generally less than about 400 grams per square meter. Generally, the weight of the adhesive is at least 20 grams per square meter. Typically, the adhesive is applied in an amount of about 100 grams per square meter to about 400 grams per square meter. The limitations on the basis weight of the adhesive are important to provide the correct adhesive characteristics for applying directly to the wearer's body. If the basis weight is too high, the body-adhering absorbent article 20 will have a sticky feeling or otherwise uncomfortable feeling. If the basis weight of the adhesive is too low, there can be insufficient adhesion to the body of the wearer.

The body adhesive is generally applied in a manner that is symmetrical about the longitudinal centerline of the body-adhering absorbent article 20. This symmetrical pattern provides the wearer a balanced feel when wearing the body-adhering absorbent article 20. The symmetrical pattern also reduces the perception of any associated discomfort when the body-adhering absorbent article 20 is removed from the body.

It will be appreciated that details of the foregoing examples, given for purposes of illustration, are not to be construed as limiting the scope of this disclosure. Although only a few exemplary aspects of this disclosure have been described in detail above, those skilled in the art will readily appreciate that many modifications are possible in the examples without materially departing from the novel teachings and advantages of this disclosure. For example, features described in relation to one example can be incorporated into any other example of the disclosure.

Accordingly, all such modifications are intended to be included within the scope of this disclosure, which is defined in the following claims and all equivalents thereto. Further, it is recognized that many aspects can be conceived that do not achieve all of the advantages of some aspects, particularly of the preferred aspects, yet the absence of a particular advantage shall not be construed to necessarily mean that such an aspect is outside the scope of the present disclosure. As various changes could be made in the above constructions without departing from the scope of the disclosure, it is intended that all matter contained in the above description shall be interpreted as illustrative and not in a limiting sense.

We claim:

1. An absorbent article system comprising:
a support member having
anterior and posterior ends,
a first anchor connection zone adjacent one of the anterior and posterior ends, and
an absorbent article component connection zone between the anterior and posterior ends; and
a first anchor configured to be connectable to the first anchor connection zone, wherein one of the first anchor and the first anchor connection zone includes an anchor attachment mechanism, wherein the first anchor has a body-facing side and an attachment side, the body-facing side including a body adhesive.

2. The system of claim 1, further comprising a second anchor connection zone adjacent the other of the anterior and posterior ends, and a second anchor configured to be connectable to the second anchor connection zone, wherein one of the second anchor and the second anchor connection zone includes an anchor attachment mechanism, wherein the second anchor has a body-facing side and an attachment side, the body-facing side including a body adhesive.

3. The system of claim 2, wherein the second anchor is configured to be connected to the support member by a wearer.

4. The system of claim 1, wherein the first anchor is affixed to the support member.

5. The system of claim 1, further comprising an absorbent article component, wherein one of the absorbent article component and the absorbent article component connection zone includes a pad attachment mechanism.

6. The system of claim 5, wherein the absorbent article component is a standard absorbent pad having a standard pad attachment mechanism, and wherein the support member is configured to be connectable to the standard pad attachment mechanism.

7. The system of claim 1, wherein the support member is non-absorbent.

8. The system of claim 1, wherein the first anchor is non-absorbent.

9. The system of claim 1, wherein one of the first anchor connection zone and the first anchor are configured to allow the first anchor to be connected to the support member at different positions within the first anchor connection zone.

10. The system of claim 1, wherein the first anchor is reusable.

11. The system of claim 1, wherein the support member is reusable.

12. The system of claim 1, further comprising a second support member configured to be connectable to the first anchor and to an absorbent article component.

13. The system of claim 1, wherein the anchor attachment mechanism includes an adhesive.

14. The system of claim 1, wherein the anchor attachment mechanism includes a mechanical fastener.

15. The system of claim 1, wherein the support member is elastomeric.

16. An absorbent article system comprising:
a support member having anterior and posterior ends, a first anchor connection zone adjacent one of the anterior and posterior ends, a second anchor connection zone adjacent the other of the anterior and posterior ends, and an absorbent article component connection zone between the anterior and posterior ends;
a first anchor configured to be connectable to the first anchor connection zone, wherein one of the first anchor and the first anchor connection zone includes an anchor attachment mechanism, wherein the first anchor has a body-facing side and an attachment side, the body-facing side including a body adhesive;
a second anchor configured to be connectable to the second anchor connection zone, wherein one of the second anchor and the second anchor connection zone includes an anchor attachment mechanism, wherein the second anchor has a body-facing side and an attachment side, the body-facing side including a body adhesive, wherein one of the first and second anchors is configured to be connected to the support member by a wearer; and
an absorbent article component, wherein one of the absorbent article component and the absorbent article component connection zone includes a pad attachment mechanism, and wherein one of the absorbent article component and the absorbent article component connection zone is configured to allow the absorbent article component to be connected to the support member by a wearer.

17. The system of claim 16, wherein the absorbent article component is a standard absorbent pad having a standard pad attachment mechanism, and wherein the support member is configured to be connectable to the standard pad attachment mechanism.

18. A system for converting a garment-attached absorbent article to a body-attached absorbent article, the system comprising:
a support member having anterior and posterior ends, a first anchor connection zone adjacent one of the anterior and posterior ends, a second anchor connection zone adjacent the other of the anterior and posterior ends, and an absorbent article component connection zone between the anterior and posterior ends, the absorbent article component connection zone configured to allow the garment-attached absorbent article to be connected to the support member by a wearer;
a first anchor configured to be connectable to the first anchor connection zone, wherein one of the first anchor and the first anchor connection zone includes an anchor attachment mechanism, wherein the first anchor has a body-facing side and an attachment side, the body-facing side including a body adhesive.

19. The system of claim 18, wherein one of the first anchor connection zone and the first anchor are configured to allow the first anchor to be connected to the support member at different positions within the first anchor connection zone.

20. The system of claim 18, further comprising a second anchor configured to be connectable to the second anchor connection zone, wherein one of the second anchor and the second anchor connection zone includes an anchor attachment mechanism, wherein the second anchor has a body-facing side and an attachment side, the body-facing side including a body adhesive.
